(19) United States
(12) Patent Application Publication
(10) Pub. No.: US 2022/0288114 A1
(43) Pub. Date: Sep. 15, 2022

(54) MULTI-AGENT OCULAR FORMULATIONS AND TREATMENT METHODS

(71) Applicant: University of Utah Research Foundation, Salt Lake City, UT (US)

(72) Inventors: Randon Michael Burr, Salt Lake City, UT (US); Balamurali K. Ambati, Sandy, UT (US); Sarah A. Molokhia, Sandy, UT (US)

(73) Assignee: University of Utah Research Foundation, Salt Lake City, UT (US)

(21) Appl. No.: 17/626,469

(22) PCT Filed: Jul. 13, 2020
(86) PCT No.: PCT/US2020/041837

§ 371 Date: Jan. 11, 2022

Related U.S. Application Data
(60) Provisional application No. 62/873,121, filed on Jul. 11, 2019.

(51) Int. Cl. A61K 33/34; A61K 9/00; A61K 31/46; A61K 31/216; A61K 31/5513; A61K 31/522; A61P 27/10

(52) U.S. Cl. CPC A61K 33/34; A61K 9/0048; A61K 31/46; A61K 31/216; A61K 31/5513; A61K 31/522; A61P 27/10

(57) ABSTRACT

Opthalmic compositions are disclosed and described herein. In some embodiments, an ophthalmic composition can include as active agents a copper-containing agent and a secondary therapeutic agent in combination with a pharmaceutically acceptable carrier. The active agents can be present in amounts sufficient to treat myopic progression during a treatment period.

### RELATED APPLICATIONS

This application claims the benefit of U.S. Provisional Application Ser. No. 62/873,121, filed on Jul. 11, 2019, which is incorporated herein by reference.

### BACKGROUND

Myopia is a common vision problem that is estimated to affect nearly a quarter of the world's population with rates increasing over the past 50 years. In myopia, distant objects appear blurry while close objects can appear normal. Some of the complications that can result from myopia include retinal detachment, cataracts, and glaucoma. A combination of genetic and environmental factors can lead to myopia with some risk factors including family history, work that involves focusing on closely positioned objects, and too much time spent indoors. The mechanism causing myopia can include an eyeball that is too long or less frequently a lens that is too strong or a cornea that is too curved.

### BRIEF DESCRIPTION OF THE DRAWINGS

FIG. 1A is a graph of refractive error for treated eyes in guinea pigs during a 60-day treatment regimen with atropine sulfate monohydrate. FIG. 1B is a graph of refractive error for control eyes in guinea pigs during a 60-day treatment regimen.

FIG. 1C is a graph of refractive error for treated eyes and control eyes in guinea pigs during a 60-day treatment regimen with atropine sulfate monohydrate.

FIG. 2 is a graph showing increased corneal strength in response to copper sulfate treatment.

FIG. 3 is a graph of refractive error for treated eyes (OD) and untreated eyes (OS) in rabbits during a 60-day treatment regimen with copper sulfate pentahydrate.

FIG. 4 is a graph of lysinonorleucine (LNL) concentration in treated eyes (copper sulfate pentahydrate), control eyes, and vehicle eyes or rabbits after a 6-week treatment regimen.

FIG. 5A is a graph of refractive error for treated eyes in guinea pigs during a 14-day treatment regimen with copper.

FIG. 5B is a graph of refractive error for treated eyes in guinea pigs during a 14-day treatment regimen with atropine sulfate monohydrate and copper.

FIG. 5C is a graph of refractive error for control eyes in guinea pigs during a 14-day treatment regimen.

These drawings are provided to illustrate various aspects of the invention and are not intended to be limiting of the scope in terms of dimensions, materials, configurations, arrangements or proportions unless otherwise limited by the claims.

### DESCRIPTION OF EMBODIMENTS

Although the following detailed description contains many specifics for the purpose of illustration, a person of ordinary skill in the art will appreciate that many variations and alterations to the following details can be made and are considered to be included herein.

Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a cell” includes a plurality of such cells.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

Occurrences of the phrase “in one embodiment,” or “in one aspect,” herein do not necessarily all refer to the same embodiment or aspect.

As used herein, the terms “therapeutic agent,” “active agent,” and the like can be used interchangeably and refer to an agent that can have a beneficial or positive effect on a subject when administered to the subject in an appropriate or effective amount. In one aspect, the therapeutic or active agent can be a cross-linking agent which stimulates corneal cross-linking, either directly, or indirectly. In one embodiment, the cross-linking agent can be a copper-containing compound.

As used herein, the term “secondary therapeutic agent,” “secondary therapeutic,” “secondary active agent,” “supplemental therapeutic agent,” “supplemental therapeutic,” “supplemental active agent,” and the like can be used interchangeably and refer to a therapeutic or active agent that is different from, and provided in addition to, a cross-linking agent, and which has a mechanism of action which does not directly or indirectly impact corneal cross-linking. In one example, without limitation, the secondary therapeutic agent can have a mechanism of action that includes an activity that positively impacts a myopic condition, including control, prevention, or correction of myopia, such as reducing axial length growth, choroidal thickness, or lenticular power.

As used herein, an “effective amount” of an agent is an amount sufficient to accomplish a specified task or function desired of the agent. A “therapeutically effective amount” of a composition, drug, or agent refers to a non-toxic, but sufficient amount of the composition, drug, or agent, to achieve therapeutic results in treating or preventing a condition for which the composition, drug, or agent is known or intended to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician, veterinarian, or other qualified medical personnel using evaluations known in the art, it is recognized that individual variation and response to treatments may make the achievement of therapeutic effects a somewhat subjective decision. The determination of an effective amount or therapeutically effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986).

As used herein, a “dosing regimen” or “regimen” such as “treatment dosing regimen,” or a “prophylactic dosing regimen” refers to how, when, how much, and for how long a dose of an active agent or composition can or should be administered to a subject in order to achieve an intended treatment or effect.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate or eliminate symptoms associated with a condition present in a subject, or can be prophylactic, (i.e. to prevent or reduce the occurrence of the symptoms in a subject). Such prophylactic treatment can also be referred to as prevention of the condition.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients. Compositions can take nearly any physical state, including solid, liquid (e.g. solution), or gas. Furthermore, the term “dosage form” can include one or more formulation(s) or composition(s) provided in a format for administration to a subject. For example, an injectable dosage form would be a formulation or composition prepared in a manner that is suitable for administration via injection.

In one example, the term “secondary therapeutic” can refer to the secondary therapeutic agent and any functionally similar compound, including, without limitation, analogues, homologues, isomers, metabolites, derivatives, and the like.

As used herein, a “subject” refers to an animal. In one aspect the animal may be a mammal. In another aspect, the mammal may be a human.

As used herein, “pharmaceutically acceptable carrier” and “carrier” may be used interchangeably, and refer to any inert and pharmaceutically acceptable material that has substantially no biological activity, and makes up a substantial part of the formulation. The carrier may be polymeric, such as an adhesive, or non-polymeric, and is generally admixed with other components of the composition (e.g., drug, binders, fillers, permeation enhancers, anti-irritants, emollients, lubricants, and the like) to comprise the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 micrograms to about 80 micrograms” should also be understood to provide support for the range of “50 micrograms to 80 micrograms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Myopia is expected to affect half of the global population by the year 2050. The mechanism causing myopia can include an eyeball that is too long or less frequently a lens that is too strong. When an eyeball is too long, incoming light rays can be focused at a point in front of the retina rather than on the surface of the retina. In degenerative or pathological myopia, the risk of retinal detachment, bleeding in the eye, cataracts can be significantly increased.

While the underlying mechanism causing myopia can be an elongated eyeball, various causes have been hypothesized and researched. Scientists have researched approximately 24 genetic risk factors for myopia, including genes involved in nerve cell function, metabolism, and eye development. Individuals carrying a large number of the genetic risk factors can have a tenfold increased risk of myopia.

Environmental factors can also play a role in the development of myopia. Some of the environmental factors that have been commonly associated with myopia include lack of time spent outdoors and increasing amounts of time doing near-work (e.g., reading, writing, and computer-work). Research has indicated that time spent outdoors during the time myopia typically develops (children between the ages of 6 and 14) can reduce the risk of developing myopia independently of time doing near-work. However, once myopia has begun to develop, time spent outdoors may not slow its progression. Therefore, improved treatments for myopia (especially children between the ages of 6 and 14) are desired.

This disclosure is directed to multi-agent ocular compositions and related methods, such as for treating myopia and/or preventing progression of myopia in a subject. In some examples, treating myopia and/or preventing progression of myopia in a subject can include administration of a therapeutically effective amount of a multi-agent, ophthalmic dosage form, such as a topical dosage form.

In one example, an ophthalmic composition, an ophthalmic dosage form, or ophthalmic medicament is described herein. The ophthalmic composition, ophthalmic dosage form, or ophthalmic medicament can include an amount of copper-containing agent, or cross-linking agent, that is sufficient to increase lysyl oxidase activity in an eye of a subject or otherwise increase cross-linking in the cornea of the subject. The ophthalmic composition, ophthalmic dosage form, or ophthalmic medicament can further include an amount of a secondary therapeutic agent that is sufficient to reduce myopia, for example, by one or more of reducing axial length growth, choroidal thickness, or lenticular power. The ophthalmic composition, ophthalmic dosage form, or ophthalmic medicament can further include a pharmaceutically acceptable carrier. In some examples, the ophthalmic dosage form can be an ophthalmic composition formulated as a topical eye drop that can be dispensed from a container in a drop-wise manner at a drop volume of about 5 μl to about 50 μl. In some examples, a method is described for using such an ophthalmic composition, ophthalmic dosage form, or ophthalmic medicament. The method can include administering a therapeutically effective amount of an ophthalmic composition, ophthalmic dosage form, or ophthalmic medicament, as described herein, during a treatment period.

In some examples, myopia can include a cornea with reduced rigidity. The ophthalmic compositions, ophthalmic dosage forms, and ophthalmic medicaments described herein can increase lysl oxidase activity in an eye of a subject. The increased lysyl oxidase activity can increase corneal rigidity and provide other benefits that can treat or prevent progression of myopia in an individual. In another example, the ophthalmic compositions, ophthalmic dosage forms, and ophthalmic medicaments described herein can reduce myopia by one or more of: reducing axial length growth, choroidal thickness, or lenticular power.

In one example, a copper-containing agent can be a co-factor for lysyl oxidase (LOX), which is an enzyme that promotes the formation of various types of collagen cross-links. Therefore, a copper-containing agent (e.g., a copper-containing salt, a copper-containing compound, a copper-containing chelate, and the like) can promote an increase in collagen bonds and a concomitant enhancement in the biomechanical properties of the cornea. Because low corneal rigidity can be associated with myopia and supplementation with a copper-containing agent can enhance the rigidity of the cornea, treatment with a copper-containing agent can provide a non-invasive and low-cost way of treating or preventing the progression of myopia.

In some examples, an ophthalmic composition, ophthalmic dosage form, or ophthalmic medicament is described herein that can include an amount of a copper-containing agent that can increase LOX activity in an eye of a subject or otherwise increase corneal cross-linking. A variety of copper-containing agents can be used, such as a copper-containing salt, a copper-containing compound, a copper-containing chelate, or the like. Copper salts can include one or more of: copper sulfates, copper carbonates, copper acetates, copper chlorides, copper bromides, copper fluorides, copper nitrates, copper hydroxides, copper iodides, copper perchlorates, copper molybdates, copper thiocyanates, copper tartrates, copper tetrafluoroborates, copper selenides, copper pyrophosphates, hydrates thereof, the like, or any combination thereof. Other copper carriers can include one or more of: GHK-copper, tetra-amine copper sulfate, copper-histidine, copper-glycinate, copper-gluconate, hydrates thereof, the like, or any combinations thereof.

Thus, the copper-containing agent can be any suitable copper-containing agent that can provide a therapeutically effective amount of copper. The therapeutically effective amount can be sufficient to: (a) increase corneal LOX activity in the eye; (b) increase collagen cross-linking as compared to collagen cross-linking prior to treatment; (c) increase the biomechanical strength of the cornea compared to the biomechanical strength of the cornea prior to treatment; or (d) decrease the diopter of the cornea in the treated eye as compared to the diopter of the cornea prior to treatment.

A therapeutically effective amount of a copper-containing agent can be based upon the amount of copper carried by the copper-containing agent. In some examples, the copper-containing agent can provide a composition having copper levels less than about 0.625 mg/ml, about 0.05 mg/ml, about 0.02 mg/ml, about 0.005 mg/ml, or about 0.002 mg/ml, but that are still effective at increasing lysyl oxidase activity. In another example (e.g., when a copper-containing agent is administered with a secondary therapeutic agent), the copper-containing agent can provide a composition having copper levels less than about 0.0625 mg/ml, about 0.005 mg/ml, about 0.002 mg/ml, about 0.0005 mg/ml, or about 0.0002 mg/ml, or about 0.00002 mg/ml. It can be important to keep the copper level sufficiently low to avoid copper-induced toxicity, while maintaining a sufficient amount of bioavailable copper to increase lysyl oxidase activity.

Thus, the therapeutically effective amount of the copper-containing agent can be determined based on the type of delivery vehicle, the type of copper-containing agent, the desired delivery duration, etc. For example, depending on the how the composition is formulated, the composition can include an amount of copper from about 0.000005 mg/ml or 0.00005 mg/ml to about 5 mg/ml or about 50 mg/ml. In other examples, the composition can include an amount of copper from about 0.000006 mg/ml to about 0.007 mg/ml, from about 0.00006 mg/ml to about 0.07 mg/ml, from about 0.0006 mg/ml to about 0.007 mg/ml, from about 0.0005 mg/ml to about 0.03 mg/ml, from about 0.01 mg/ml to about 5 mg/ml, or from about 0.001 to about 0.005 mg/ml. In some additional examples, the composition can include an amount of copper from about 0.0001 mg/ml to about 0.05 mg/ml, about 0.00025 mg/ml to about 0.015 , about 0.0005 mg/ml to about 0.00075 mg/ml, or about 0.0008 mg/ml to about 0.0011 mg/ml. Thus, in some examples, the therapeutically effective amount can be defined as the amount of copper included in the composition. For example, an amount of 0.0025 mg/ml of copper (II) sulfate, pentahydrate, provides the composition with a copper content of about 0.000636 mg/ml copper. This is because the atomic weight of copper (II) sulfate, pentahydrate is about 249.677 g/mol, but only about 63.5 g/mol or about 25% of the agent is copper itself. Thus, the therapeutically effective amount can be determined based on the copper content provided by the copper-containing agent rather than the amount of copper-containing agent itself. As an alternative example, an amount of 0.0018 mg/ml of copper (II) acetate, anhydrous, provides the composition with a copper content of about 0.00063 mg/ml.

Alternatively, the therapeutically effective amount can be defined as a wt % of the copper-containing agent in the composition. Again, depending on how the composition is formulated, the therapeutically effective amount of the copper-containing agent can be an amount from about 0.00001 wt % or 0.0001 wt % to about 5 wt %, 10 wt %, or 15 wt %. In some examples, the therapeutically effective amount of the copper-containing agent can be from about 0.05 wt % to about 15 wt %, from about 0.01 wt % to about 10 wt %, or from about 0.005 wt % to about 5 wt %. In other examples, the therapeutically effective amount of the copper-containing agent can be an amount from about 0.00001 wt % to about 0.0001 wt %, from about 0.0001 wt % to about 0.0005 wt %, from about 0.0001 wt % to about 0.0002 wt %, from about 0.0002 wt % to about 0.0003 wt %, or from about 0.0003 wt % to about 0.0004 wt %. In yet other examples, the therapeutically effective amount of the copper-containing agent can be an amount from about 0.001 wt % to about 0.01 wt % or about 0.003 wt % to about 0.008 wt %. In yet other examples, the therapeutically effective amount of the copper-containing agent can be an amount from about 0.01 wt % to about 0.1 wt %, or from about 0.03 wt % to about 0.08 wt %. It is noted that these weight percentages are calculated based on copper (II) sulfate, anhydrous. Thus, where an alternative copper-containing agent is employed, the weight percentages can be converted accordingly.

In another example (e.g., when a copper-containing agent is administered with a secondary therapeutic agent), the therapeutically effective amount of the copper-containing agent can be an amount from about 0.000001 wt % or 0.00001 wt % to about 5 wt %, 10 wt %, or 15 wt %. In some examples, the therapeutically effective amount of the copper-containing agent can be from about 0.005 wt % to about 15 wt %, from about 0.001 wt % to about 10 wt %, or from about 0.0005 wt % to about 5 wt %. In other examples, the therapeutically effective amount of the copper-containing agent can be an amount from about 0.000001 wt % to about 0.00001 wt %, from about 0.00001 wt % to about 0.00005 wt %, from about 0.00001 wt % to about 0.00002 wt %, from about 0.00002 wt % to about 0.00003 wt %, or from about 0.00003 wt % to about 0.00004 wt %. In yet other examples, the therapeutically effective amount of the copper-containing agent can be an amount from about 0.0001 wt % to about 0.001 wt % or about 0.0003 wt % to about 0.0008 wt %. In yet other examples, the therapeutically effective amount of the copper-containing agent can be an amount from about 0.001 wt % to about 0.01 wt %, or from about 0.003 wt % to about 0.008 wt %.

The bioavailability of copper can vary from one copper-containing agent to another copper-containing agent. The acidity or basicity (e.g., pH) and other compositional factors of the copper-containing agent can also provide variation in bioavailability of copper. Further, the release rate of the copper from a particular dosage form can be adjusted based on the particular copper-containing agent employed in the dosage form. For example, in some cases a less soluble copper-containing agent (e.g., copper fluoride, copper hydroxide, copper carbonate, and the like) can be used to prolong the release of the copper-containing agent from the composition.

The copper-containing agent can also be administered with a therapeutically effective amount of a secondary therapeutic agent. In one example, a secondary therapeutic agent can include one or more of: atropine, homatropine, cyclopentolate, pirenzepine, 7-methylxanthanine, the like, or any combinations thereof. In another example, a secondary therapeutic agent can include one or more of: scopolamine, tropicamide, benztropine, biperiden, trihexyphenidyl, oxybutynin, tolterodine, solifenacin, dicyclomine, darifenacin, or any combinations thereof. In another example, a secondary therapeutic agent can include one or more of: glycopyrrolate, ipratropium bromide, tiotropium bromide, or any combinations thereof. In another example, the secondary therapeutic agent can include one or more antimuscarinic agents (i.e. anticholingergics) including one or more of: antipsychotics (e.g., clozapine or quetiapine), chlorpheniramine, selective serotonin reuptake inhibitors (e.g., citalopram or sertraline), dimenhydrinate, diphenhydramine, doxepin, doxylamine, glycopyrrolate, glycopyrronium, hyoscyamine, ipratropium, orphenadrine, oxitropium, promethazine, propantheline bromide, tolterodine, tiotropium, tricyclic antidepressants, or any combinations thereof. In another example, atropine can include one or more of: tropoyltropan-3β-ol, tropoylgranatan-3α-ol, tropoylgranatan-3β-ol, tropoyltropan-3α-ol, or any combination thereof. For purposes of this disclosure, “atropine” can include any of the foregoing agents, including any combinations, analogues, homologues, isomers, derivatives, salts, or metabolites thereof.

Thus, in one example, the secondary therapeutic agent can be any suitable atropine-containing agent that can provide a therapeutically effective amount of atropine. The therapeutically effective amount of atropine can be sufficient to: reduce myopia by reducing one or more of: axial length growth, choroidal thickness, lenticular power, or any combination thereof. A therapeutically effective amount of a secondary therapeutic agent can be based upon the amount of atropine carried by the secondary therapeutic agent. In some examples, the secondary therapeutic agent can provide a composition having atropine levels less than about 20.0 mg/ml, about 2.0 mg/ml, about 0.2 mg/ml, about 0.02 mg/ml, about 0.005 mg/ml, or about 0.001 mg/ml, but that are still effective at reducing myopia. It can be important to keep the atropine level sufficiently low to avoid toxicity, while maintaining a sufficient amount of bioavailable atropine to reduce myopia.

Thus, the therapeutically effective amount of the secondary therapeutic agent can be determined based on the type of delivery vehicle, the type of secondary therapeutic agent, the desired delivery duration, and the like. For example, depending on the how the composition is formulated, the composition can include an amount of atropine from about 0.001 mg/ml to about 5 mg/ml or about 20 mg/ml. In other examples, the composition can include an amount of atropine from about 0.005 mg/ml to about 0.05 mg/ml, from about 0.005 mg/ml to about 0.050 mg/ml, from about 0.050 mg/ml to about 0.5 mg/ml, from about 0.5 mg/ml to about 5 mg/ml, or from about 0.01 mg/ml to about 0.1 mg/ml. In some additional examples (e.g., when the secondary therapeutic agent is administered with a copper-containing agent), the composition can include an amount of atropine from about 0.0001 mg/ml to about 0.005 mg/ml, about 0.001 mg/ml to about 0.015, about 0.015 mg/ml to about 0.75 mg/ml, or about 0.75 mg/ml to about 7.5 mg/ml.

Thus, in some examples, the therapeutically effective amount can be defined as the amount of atropine included in the composition. Thus, the therapeutically effective amount can be determined based on the atropine content provided by the secondary therapeutic agent rather than the amount of secondary therapeutic agent itself.

Alternatively, the therapeutically effective amount can be defined as a wt % of the secondary therapeutic agent in the composition. Again, depending on how the composition is formulated, the therapeutically effective amount of the secondary therapeutic agent can be an amount from about 0.0001 wt % or 0.001 wt % to about 1 wt %, 5 wt %, 10 wt %, or 15 wt %. In some examples, the therapeutically effective amount of the secondary therapeutic agent can be from about 0.0005 wt % to about 2% wt %, from about 0.001 wt % to about 1 wt %, or from about 0.005 wt % to about 5 wt %. In other examples, the therapeutically effective amount of the secondary therapeutic agent can be an amount from about 0.00001 wt % to about 0.0001 wt %, from about 0.0001 wt % to about 0.0005 wt %, from about 0.0001 wt % to about 0.0002 wt %, from about 0.0002 wt % to about 0.0003 wt %, or from about 0.0003 wt % to about 0.0004 wt %. In yet other examples, the therapeutically effective amount of the secondary therapeutic agent can be an amount from about 0.001 wt % to about 0.01 wt % or about 0.003 wt % to about 0.008 wt %. In yet other examples, the therapeutically effective amount of the secondary therapeutic agent can be an amount from about 0.01 wt % to about 0.1 wt %, or from about 0.03 wt % to about 0.08 wt %. In some additional examples (e.g., when the secondary therapeutic agent is administered with a copper-containing agent), the therapeutically effective amount of the secondary therapeutic agent can be from 0.0001 wt % to about 2 wt %. It is noted that these weight percentages are calculated based on atropine sulfate monohydrate. Thus, where an alternative secondary therapeutic agent is employed, the weight percentages can be converted accordingly.

In another example, the therapeutically effective amount of a cross-linking agent and the therapeutically effective amount of the secondary therapeutic agent can comprise a ratio of an amount of cross-linking agent to an amount of secondary therapeutic agent ranging from about 2:1 to about 1:700. In one example, the ratio can range from about 1:5 to about 1:100. In another example, the ratio can range from about 1:5 to about 1:25. In another example, the ratio can range from about 1:10 to about 1:20.

In another example, when the cross-linking agent is a copper-containing agent and the secondary therapeutic agent is atropine, the ratio of amount of copper-containing agent to an amount of atropine can range from about 2:1 to about 1:700. In one example, the ratio can range from about 1:5 to about 1:100. In another example, the ratio can range from about 1:5 to about 1:25. In another example, the ratio can range from about 1:10 to about 1:20.

The bioavailability of atropine can vary from one secondary therapeutic agent to another secondary therapeutic agent. The acidity or basicity (e.g., pH) and other compositional factors of the secondary therapeutic agent can also provide variation in bioavailability of atropine. Further, the release rate of the atropine from a particular dosage form can be adjusted based on the particular secondary therapeutic agent employed in the dosage form. For example, in some cases a less soluble secondary therapeutic agent can be used to prolong the release of the secondary therapeutic agent from the composition.

The copper-containing agent and the secondary therapeutic agent can also be administered with an additional active agent. The additional active agent can include one or more of: riboflavin, rose bengal, hydroxylysine, a calcium-containing agent, a magnesium-containing agent, a silver-containing agent, an aluminum-containing agent, a zinc-containing agent, iron-containing agent, acai extract, decorin, biglycan, keratocan, lumican, mimican, fibromodulin, type VI collagen, type X collagen, type XII collagen, type XIV collagen, or any combinations thereof.

In some examples, one or more alternative cross-linking agents can be administered instead of a copper-containing agent. For example, in some cases an alternative cross-linking agent can be or include any divalent or multivalent ion or compound that is suitable to induce or facilitate cross-linking in the cornea. In some examples, the cross-linking agent can be or include a metal ion, such as an alkaline earth metal, a transition metal, a to post-transition metal, or combinations thereof, for example. In some examples, the cross-linking agent can be or include a cation. In some specific examples, the cross-linking agent can be or include a divalent metal ion, such as magnesium, iron, zinc, or the like.

In some examples, alternative cross-linking agents can include, but are not limited to: a calcium-containing agent, a magnesium-containing agent, a silver-containing agent, an aluminum-containing agent, a zinc-containing agent, iron-containing agent, or other suitable cross-linking agent. Some specific, but non-limiting, examples of cross-linking agents can include acai extract, decorin, copper (II) sulfate, or combinations thereof.

In some examples, a combination of a therapeutically effective amount of a cross-linking agent and/or a therapeutically effective amount of a secondary therapeutic agent can be used in a method of treating and/or preventing progression of conditions affected by insufficient cross-linking (e.g. a cross-linking, such as collagen cross-linking, deficiency). In addition to myopia, other conditions can include, but are not limited to: keratoconus, ectasia, keratectasia, astigmatism, hyperopia, keratitis, presbyopia, bullous keratopathy, cogan syndrome, corneal ulcer, interstitial keratitis, keratoconjunctivitis sicca, keratomalacia, peripheral ulcerative keratitis, phlyctenular keratoconjunctivitis, superficial punctate keratitis, the like, and combinations thereof.

In other examples, the secondary therapeutic agent can be used in a method of treating and/or preventing myopia, keratoconus, ectasia, keratectasia, astigmatism, hyperopia, keratitis, presbyopia, bullous keratopathy, cogan syndrome, corneal ulcer, interstitial keratitis, keratoconjunctivitis sicca, keratomalacia, peripheral ulcerative keratitis, phlyctenular keratoconjunctivitis, superficial punctate keratitis, the like, and combinations thereof, as the primary therapeutic agent. In these examples, the cross-linking agent can provide a supporting role to the secondary therapeutic agent.

The copper-containing agent and the secondary therapeutic agent can be provided in a pharmaceutically acceptable carrier. The pharmaceutically acceptable carrier can be formulated in a variety of ways to deliver the copper-containing agent and the secondary therapeutic agent. Non-limiting examples can include solutions, suspensions, emulsions, gels, hydrogels, thermo-responsive gels, formulation for subconjunctival injection, formulation for sub-tenon's injection, depots, films, sustained-delivery matrixes, contact lenses, pledgets, the like, or a combination thereof.

In some examples, the composition can be formulated for passive delivery to the eye. In other examples, the composition can be formulated for active delivery to the eye, such as iontophoresis, electroporation, sonoporation, or the like. In one specific example, the formulation can be an ophthalmic drop. In some examples, the composition can be formulated as a copper-eluting contact lens or a secondary therapeutic agent eluting contact lens, such as a soft lens, a toric lens, a hard lens, a scleral lens, the like, or combination thereof. In some examples, the composition can be formulated as a sustained-delivery matrix for placement in contact with an ocular surface, such as in a cul-de-sac, conjunctiva, tenon's capsule, or the like.

In some examples, as mentioned in inventor's U.S. patent application Ser. Nos. 16/083,865 filed Sep. 10, 2018 and 16/960,077 filed Jul. 4, 2020, which are incorporated herein by reference, a pharmaceutically acceptable carrier can include, without limitation, one or more of a solubilizing agent, a tonicity agent, a pH adjuster, a thickener or gelling agent, a polymer or polymeric matrix, a preservative, water, the like, and combinations thereof.

Solubilizing agents can include, without limitation, one or more of: phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, BSS Plus, Ringer's lactate solution, normal saline (i.e. 0.9% saline), ½normal saline, the like, or combinations thereof. Solubilizing agents can be present in the pharmaceutically acceptable carrier in various amounts depending on the particular formulation, method of treatment, and the like.

Tonicity agents can include, without limitation, one or more of: the solubilizing agents previously listed, sodium chloride, potassium chloride, calcium chloride, magnesium chloride, mannitol, sorbitol, dextrose, glycerin, propylene glycol, ethanol, trehalose, the like, or combinations thereof. The tonicity agent can be used to provide an appropriate tonicity of the formulation. In one aspect, the tonicity of the formulation can be from about 250 to about 350 milliosmoles/liter (mOsm/L). In another aspect, the tonicity of the formulation can be from about 270 to about 330 mOsm/L. Tonicity agents can be present in the pharmaceutically acceptable carrier in various amounts depending on the particular formulation, method of treatment, and the like. In some examples, pH adjusters can include, without limitation, a number of acids, bases, and combinations thereof, such as: hydrochloric acid, phosphoric acid, citric acid, sodium hydroxide, potassium hydroxide, calcium hydroxide, and the like. The pH adjusters can be used to provide an appropriate pH for the formulation. Where applicable, in one aspect, the pH can be from about 5.5 to about 8.5. In another aspect, the pH can be from about 5.8 to about 7.8. In another aspect, the pH can be from about 6.5 to about 7.8. In yet another aspect, the pH can be from about 7.0 to about 7.6. The pH adjusters can be present in the pharmaceutically acceptable carrier in various amounts depending on the particular formulation, method of treatment, and the like.

Thickeners or gelling agents can include, without limitation, one or more of: glycerol, propylene glycol, polyethylene glycol, polyvinyl alcohol, cellulose derivatives (such as methyl cellulose, carboxymethyl cellulose, hydroxypropyl cellulose, and the like) ethylvinyl alcohol, hyaluronic acid, the like, or combinations thereof. Thickeners or gelling agents can be present in the pharmaceutically acceptable carrier in various amounts depending on the particular formulation, method of treatment, and the like.

Polymers that can be used to prepare a polymer matrix for a film, contact lens or the like, can include biodegradable or non-biodegradable polymers. Polymers or polymer combinations can include, without limitation, one or more of: poly(methylmethacrylate), polyorthoesters, hydroxyethylmethacryIate, polysiloxanes, poly(lactic-co-glycolic acid) (different ratios of lactic to glycolide content and end groups such as acid or ester termination), polyvinyl alcohol, polyvinyl acetate, ethylene vinyl acetate, polyethylene glycol, polylactic acid, polyglycolic acid, hydroxypropyl methylcellulose, hydroxypropylcellulose, carboxymethylcellulose, croscarmellose, polycaprolactone, hyaluronic acid, albumin, sodium chloride block copolymers thereof, salts thereof, the like, or combinations thereof. Specific copolymers such as polylactic-polyglycolic acid block copolymers (PLGA), polyglycolic acid-polyvinyl alcohol block copolymers (PGA/PVA), hydroxypropylmethylcellulose (HPMC), polycaprolactone-polyethylene glycol block copolymers, croscarmellose, and the like can be particularly effective for biodegradable matrixes.

In some examples, the composition can include thermo-responsive polymers. Thermo-responsive polymers can include, without limitation, one or more of: poly(N-isopropyl acrylamide), poly [2-(dimethylamino)ethylmethacrylate], hydroxypropylcellulose, poly(vinyl caprolactame), polyvinyl methyl ether, polyethylene oxide, polyhydroxyethylmethacrylate, ABCBA-type pentablock polymers, chitosan, the like, or combinations thereof. Such thermo-responsive polymers can bind or can be functionalized to bind a particular copper-containing agent or a particular secondary therapeutic agent within a range of temperatures and release the copper-containing agent or secondary therapeutic agent upon changing the temperature of the surrounding environment, such as placing the composition in contact with the eye, applying a heat source to the eye after administration of the composition, or the like.

Preservatives can include, without limitation, one or more of: benzalkonium chloride (BAK), cetrimonium, sodium perborate, ethylenediaminetetraaceticacid (EDTA) and its various salt forms, chlorobutanol, and the like. Preservatives can be present in the pharmaceutically acceptable carrier in various amounts depending on the particular formulation, method of treatment, and the like.

In one example, the pharmaceutically acceptable carrier can be formulated as an ophthalmic drop and can include BSS or other suitable solubility or tonicity agent. In another example, the pharmaceutically acceptable carrier can be formulated as an ophthalmic drop and can include artificial tears (e.g. Refresh Tears®, Genteal®, Oasis Tears®, or the like). The pharmaceutically acceptable carrier can be formulated as a thin film, ointment, gellating suspension, punctal plug, or contact lens (or a coating thereon).

In one example, the ophthalmic composition can be used as an ophthalmic dosage form to administer a therapeutically effective dose of the copper-containing agent and the secondary therapeutic agent. In some examples, the ophthalmic dosage form can provide from about 0.0005 μg to about 0.5 μg of copper per administration event. In yet other examples, the ophthalmic dosage form can provide from about 0.006 μg to about 0.06 μg, about 0.01 μg to about 0.03 μg, or about 0.016 μg to about 0.044 μg of copper per administration event. In yet other examples, the ophthalmic dosage form can provide from about 0.0005 μg to about 5 μg of copper per day. In yet other examples, the ophthalmic dosage form can provide from about 0.001 μg to about 2 μg, about 0.006 μg to about 0.24 μg, about 0.01 μg to about 0.12 μg, or about 0.016 μg to about 0.18 μg of copper per day. In some additional examples (e.g., when a secondary therapeutic agent is administered with the copper-containing agent), the ophthalmic dosage form can provide from about 0.00005 μg to about 0.05 μg of copper per administration event.

In some examples, the ophthalmic dosage form can provide from about 0.05 μg to about 0.5 μg of secondary therapeutic agent per administration event. In yet other examples, the ophthalmic dosage form can provide from about 0.06 μg to about 0.6 μg, about 0.1 μg to about 0.3 μg, or about 0.16 μg to about 0.44 μg of secondary therapeutic agent per administration event. In yet other examples, the ophthalmic dosage form can provide from about 0.05 μg to about 5 μg of secondary therapeutic agent per day. In yet other examples, the ophthalmic dosage form can provide from about 0.01 μg to about 200 μg, about 0.01 μg to about 100 μg, about 0.01 μg to about 10 μg, or about 0.01 μg to about 1 μg of secondary therapeutic agent per day. In some additional examples (e.g., when the secondary therapeutic agent is administered with a copper-containing agent), the ophthalmic dosage form can provide from about 0.001 μg to about 20 μg, or about 0.001 μg to about 10 μg, about 0.001 μg to about 1 μg, or about 0.001 μg to about 0.1 μg of secondary therapeutic agent per day. It is noted that not all of the copper or secondary therapeutic agent that is provided by the dosage form necessarily becomes bioavailable, but in some examples it can.

In some examples, the ophthalmic dosage form can be used in an effective dosage regimen to provide a therapeutically effective amount of the copper-containing agent and the secondary therapeutic agent. The effective dosage regimen can include administering the ophthalmic dosage form once per day, twice per day, three times per day, four times per day, or more.

In some examples, the ophthalmic dosage form can be formulated to biodegrade to provide controlled and sustained release of the copper-containing agent or secondary therapeutic agent over a selected period of time. In other examples, the ophthalmic dosage form can be formulated to release the copper-containing agent or secondary therapeutic agent from a non-biodegradable matrix in a controlled and sustained manner. The dosage form can be formulated to release the copper-containing agent or secondary therapeutic agent over a period of hours, days, or weeks, as desired. In some examples, the dosage form can be formulated to deliver from about 0.005 mcg of copper to about 250 mcg of copper per week. In yet other examples, the dosage form can be formulated to deliver from about 0.008 mcg to about 200 mcg per week, about 0.01 mcg to about 150 mcg per week, or about 0.1 mcg to about 100 mcg per week. In some examples (e.g., when a secondary therapeutic agent is administered with the copper-containing agent), the dosage form can be formulated to deliver from about 0.0005 mcg of copper to about 25 mcg of copper per week.

In some examples, the dosage form can be formulated to deliver from about 0.1 mcg of secondary therapeutic agent to about 250 mcg of secondary therapeutic agent per week. In yet other examples, the dosage form can be formulated to deliver from about 0.8 mcg to about 200 mcg per week, about 0.1 mcg to about 150 mcg per week, or about 1.0 mcg to about 100 mcg per week. In some examples (e.g., when the secondary therapeutic agent is administered with a copper-containing agent), the dosage form can be formulated to deliver from about 0.01 mcg of secondary therapeutic agent to about 25 mcg of secondary therapeutic agent per week The dosage form can also be formulated to have zero-order drug release kinetics.

In some examples, the dosage form can be held in or stored in a container as a pre-mixed composition that is ready to administer without further dilution or preparation. In some embodiments, a single container can hold a volume or amount of the composition that is adequate for a single dose or multiple doses.

In some examples, a container can be made of, without limitation, one or more of: glass, polypropylene, polyethylene, polycarbonate, polyvinylchloride, the like, or a combination thereof. In some examples, the container can have a volume of from about 0.5 ml to about 50 ml. In another aspect, the container can have a volume of from about 1 ml to about 30 ml, about 5 ml to about 20 ml, or about 3 ml to about 15 ml. In one aspect, the container can hold a single dose or a plurality of doses of the therapeutic composition or dosage form. In some examples, the container can be a vial, a bottle, a blister pack, a sachet, or the like.

In some examples, about 0.005 mg to about 1 mg of the copper-containing agent can be included in the container. In yet other examples, about 0.01 mg to about 0.5 mg of the copper-containing agent can be included in the container. In some examples, about 0.001 mg to about 0.5 mg of copper can be included in the container. In some examples, about 0.005 mg to about 0.2 mg of copper can be included in the container. In another example (e.g., when the copper-containing agent is administered with a secondary therapeutic agent), about 0.0005 mg to about 0.1 mg of the copper-containing agent can be included in the container.

In some examples, about 0.05 mg to about 1 mg of the secondary therapeutic agent can be included in the container. In yet other examples, about 0.1 mg to about 0.5 mg of the secondary therapeutic agent can be included in the container. In some examples, about 0.01 mg to about 0.5 mg of secondary therapeutic agent can be included in the container. In some examples, about 0.05 g to about 0.2 mg of secondary therapeutic agent can be included in the container.

In one example, the dosage form can be a topical ophthalmic dosage form that is formulated as an eye drop and carried in a container adapted to dispense the composition in a drop-wise manner at a drop volume of from about 5 μl to about 50 μl. For example, the container can be adapted to dispense the ophthalmic composition at a drop volume of from about 5 μl to about 50 μl, such as about 15 μl, about 20 μl, about 25 μl, about 30 μl, about 35 μl, about 40 μl, about 45 or about 50 μl. In some specific examples, the drop volume can be from about 15 μl to about 40 about 5 μl to about 30 μl, about 20 μl to about 30 μl, about 25 μl to about 35 μl, or about 30 μl to about 40 μl. The dosage form can further include an administration mechanism (e.g., a syringe, a dropper, or other mechanism).

The compositions or dosage forms described herein can also be employed in a method of treating and/or preventing progression of myopia. Such a method can include administering a therapeutically effective amount of the composition or dosage form to an eye of a subject during a treatment period. In one example, the composition or dosage form can be administered at from 1 to 4 time points per day per eye in need thereof. In some examples, such as where the composition is an eye drop, the dosage amount of the composition at each time point can be from about 5 μl to about 50 μl, about 5 μl to about 30 μl, about 20 μl to about 30 μl, about 25 μl to about 35 or about 30 μl to about 40 μl. In some further examples, the composition or dosage form can be administered once every 2-5 days, once per week, once every two weeks, etc. In some cases, the composition can be formulated to have a sustained release profile of from about 2-5 days, about 1 week, about 2 weeks, or the like.

The treatment period can depend on a number of factors, such as the severity of the condition, the age of the subject at diagnosis, or the like. For example, in some cases, where the subject is a school-age child or adolescent (e.g. from about age of 5 years to about age of 18 years from the date of birth), the subject can receive treatment for a period of from about 6 months to chronic treatment, or from about one year to about 5 years, or from about two years to about three years, or other suitable period of time until a desired outcome is achieved.

In some examples, the ophthalmic composition can be administered as one or more of: an ophthalmic drop, a subconjuntival injection, a sub-tenon's injection, a topical film, a gel, a solution, a contact lens (or a coating thereon), the like, or any combination. In some examples, the topical film, gel, contact lens, or the like can be configured to biodegrade over time to provide controlled and sustained release of the copper-containing agent or secondary therapeutic agent. In some examples, the copper-containing agent or secondary therapeutic agent can be administered in connection with an ocular-shaping device (e.g., a orthokeratology-style lens). The ocular-shaping device can re-shape or otherwise hold the eye in a desired or intended shape (e.g. a non-elongated shape) to remedy the elongation of a myopic eye while improving the biomechanical strength of the eye while in the desired shape. In some examples, the use of a shaping device can further improve the outcome or rate of improvement of the method of treatment.

Generally, the methods described herein can increase collagen cross-linking in the cornea as compared to the cornea of an untreated eye. The method can increase lysinonorleucine cross-linking density, histidinyl-hydroxylysinonorleucine cross-linking density, or both. Further, the methods described herein can decrease the radial strain of the cornea by at least about 10%, 25%, or 50% as compared to the radial strain of a cornea without treatment. Additionally, the methods described herein can decrease corneal diopter of a myopic cornea as compared to the corneal diopter of an untreated myopic cornea. Additionally, the methods described herein including the secondary therapeutic agent can reduce myopia by one or more of reducing axial length growth, choroidal thickness, or lenticular power.

### EXAMPLES

### Example 1—Atropine Treatment Slows Progression of Myopia in Guinea Pigs

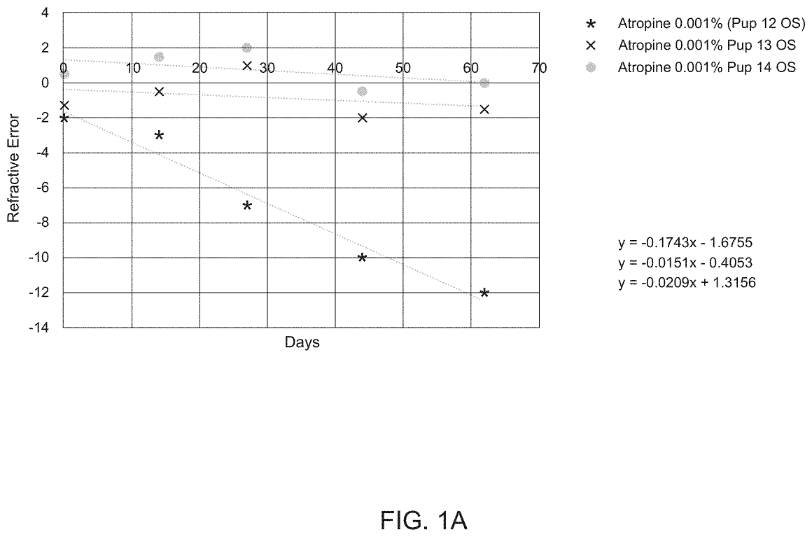

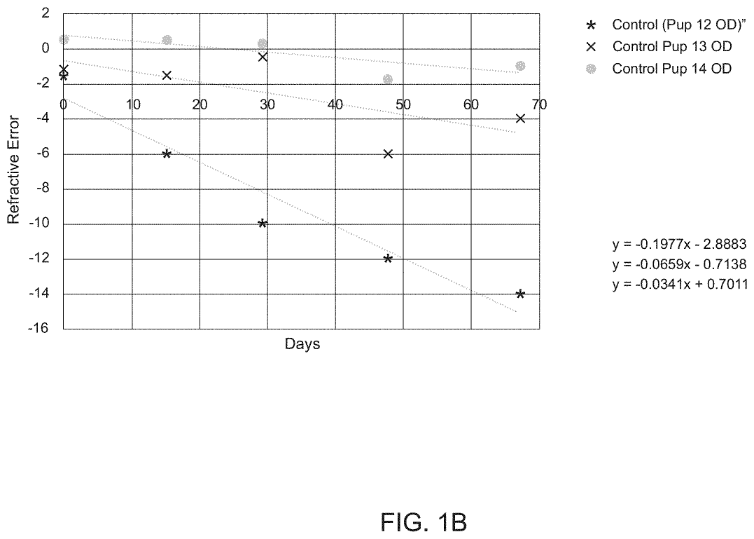

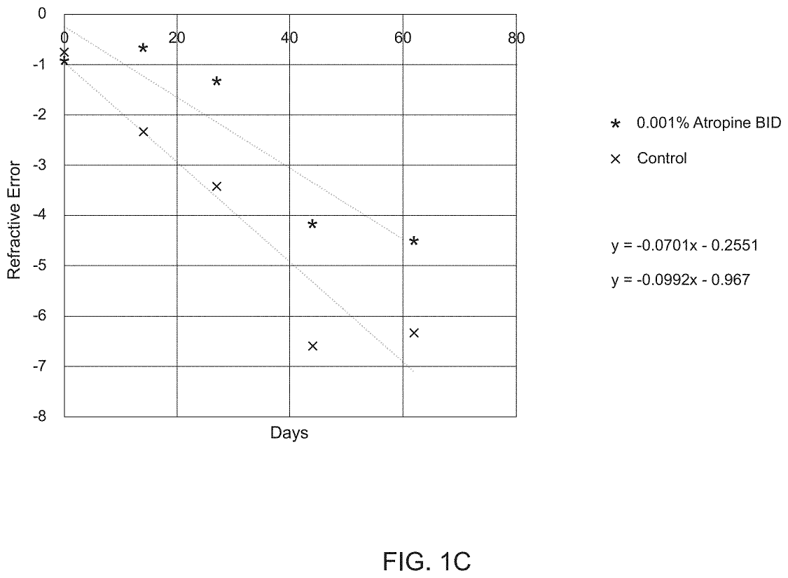

Treatment using .001% atropine sulfate monohydrate eye drops is administered at approximately 1 week of age to guinea pigs. The eye drops are administered 2 times per day for approximately 60 days. Refractive error is measured by an ophthalmologist prior to treatment on day 1 and at subsequent time-points (e.g., 14 days, 27 days, 44 days, and 62 days after the beginning of treatment) depicted in FIGS. 1A-1C. More specifically, refractive error is measured by streak retinoscopy in hand-held, awake animals in which cyloplegia had been previously induced with approximately 2 drops of 1% cyclopentolate. Stable refractive errors are typically obtained after 15 minutes when there is no pupil response. FIGS. 1A-1C present average refractive error measurements for the test subjects in each eye with linear fit.

### Example 2—Effect of Copper Sulfate on Rabbit Corneal Shape

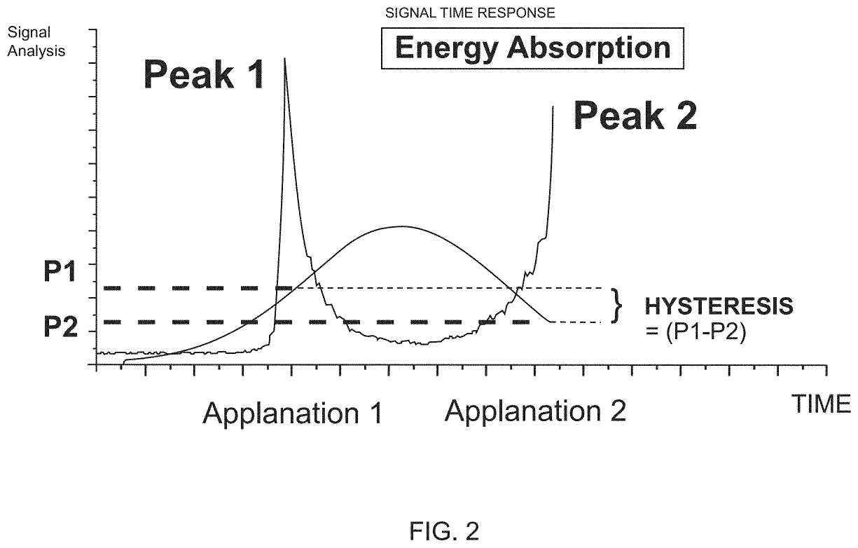

The Ocular Response Analyzer (ORA; Reichert, Inc, Buffalo, N.Y.) is a commercially available noncontact tonometer that also assesses the viscoelastic nature of the cornea with bidirectional detection of applanation events to allow in vivo measurements of corneal biomechanical response parameters under air puff loading 42. The system includes an infrared emitter and detector aligned such that applanation produces a spike on the detector with a mirror like reflection from the flattened corneal surface. Two signals are recorded, one associated with the air pressure and one associated with the infrared signal for detection of the applanation events, as seen in FIG. 2.

Two applanation pressure measurements are reported by the ORA: one while the cornea moves inward, reaching first applanation, at pressure P1, and the other after the cornea recovers from a slight concavity as it moves outward passing through a second applanation at pressure P2. Therefore, these two values, P1 and P2, indicate the pressures associated with a flattened cornea during the loading and unloading cycle, with P2<P1 for a valid measurement. The difference between P1 and P2 is termed corneal hysteresis (CH). It has been reported that CH does not change one year after crosslinking with UVA

Riboflavin, since it responds to changes in both elasticity and viscosity 43. However, significant changes were reported in the applanation signals produced, consistent with corneal stiffening. The changes included an increase in the magnitudes of both applanation spikes, Peak 1 and Peak 2. A greater value for Peak 1 or Peak 2 indicates a larger area of applanation which is associated with a stiffer response. Thus, for the current study the

ORA pressure and applanation signals were analyzed using custom software to determine the magnitudes of Peak 1 and Peak 2. ORA measurements were assessed at baseline and every week for 6-8 weeks on the same set of rabbits described in 2.4.b which included 4 groups Sol A, Sol B, and vehicle and no drops. An increase in Peak 1 and Peak 2 can be present after stiffening; therefore, one-tailed t-tests were used for statistical analysis to compare groups.

Copper sulfate generates stiffening in vivo as measured by waveform response to air puff deformation. The times at 3, 4, 5, and 6 weeks for the combined AB (treatment Sol A and treatment Sol B) treatment group were averaged for comparison against no therapy and vehicle. Peak 1 and Peak 2 were compared to both controls. For Peak 1, Group AB was significantly greater than no therapy with a p-value of 0.0244, indicating a stiffer response in the treated group. There was no substantial difference between the treated group and vehicle (p=0.0763). For Peak 2, treated group AB was significantly greater than no therapy (p=0.0098) or vehicle (p<0.0001), indicating a significantly stiffer response with treatment. There was no substantial difference between no therapy vs vehicle for either Peak 1 (p=0.5526) or Peak 2 (p=0.7049).

### Example 3—Copper Treatment Slows Progression of Myopia in Rabbits

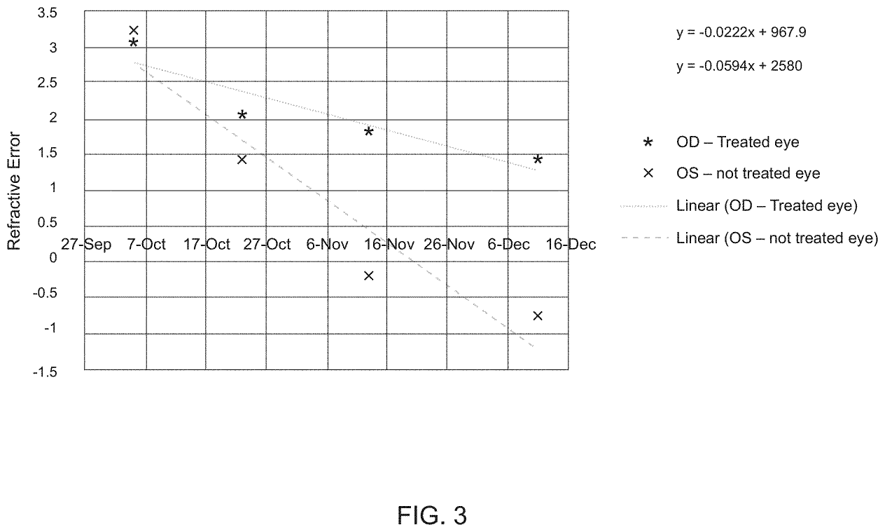

Treatment using 0.15 mg/ml copper sulfate pentahydrate in an ophthalmic vehicle is administered at approximately 6 weeks of age to rabbits. Eyedrops are administered 2 times per day for approximately 60 days. Refractive error is measured by an ophthalmologist prior to treatment on day 1 and at subsequent time-points (e.g., 14 days, 27 days, 44 days, and 62 days after the beginning of treatment) depicted in FIG. 3. More specifically, refractive error is measured by streak retinoscopy in hand-held, awake animals in which cyloplegia has been previously induced with approximately 2 drops of 1% cyclopentolate. Stable refractive errors are typically obtained after 15 minutes when there is no pupil response. FIG. 3 presents refractive error measurements for the test subjects in each eye. As depicted in FIG. 3, copper sulfate pentahydrate treatment is effective in reducing myopic progression in the treatment eye by approximately 63% compared to control.

### Example 4—Copper Treatment Increases Corneal Lysinonorleucine (LNL) Amounts In Vivo

LOX converts lysine to allysine, which is conjugated to lysine or hydroxylysine spontaneously, becoming LNL or HLNL. To test the hypothesis that copper could enhance LOX activity and hence increase the LNL amount in cornea, New Zealand white rabbits are treated using 3 groups (n=6 each). One group is treated with copper sulfate pentahydrate 0.15 mg/ml eye drops twice a day, a second with the vehicle twice a day, and a third is untreated (no eye drops) control for 6 weeks each. After six weeks each group of corneas is dissected, weighed and used for LNL analysis. Samples are reduced with NaBH4 at room temperature then washed twice with water, dried, and hydrolyzed with 6N

HCl in vacuo for 18 hours at 110° C. 6. The hydrolysates are dried to evaporate HCL, reconstituted with H2O, and re-dried to remove residual HCl. Post hydrolysis, cross-link enrichment is carried out using a cellulose mini-column method. Samples are then provided for LNL analysis by mass spectroscopy (LC/MS).

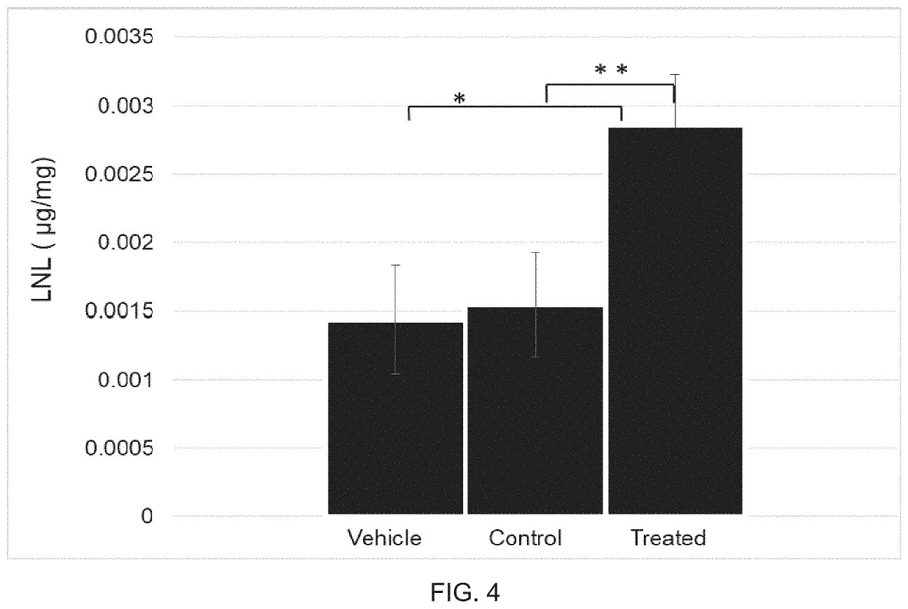

To perform liquid chromatography/mass spectroscopy (LC/MS), 1 μL of 100 μg/mL d9-Lysine is added to all samples as an internal standard. A calibration curve is created using serial dilution. Semi-quantitative mass spectral analysis is performed using a Sciex 6500 Q-Trap (Sciex, Farmington, MA). A helicon iHILIC-Fusion 2.1×100 mm column (Umeå, Sweden) is used for chromatography using 10 mM NH4OAc (Buffer A) and ACN (Buffer B). LC/MS is performed in the positive mode with a TurboIon source using optimized source conditions. Quantitative data analysis is conducted using Sciex MultiQuant software. In rabbit studies, LC-MS demonstrates significant increases in LNL crosslinks in treated rabbit cornea compared to control (no treatment) and vehicle group (FIG. 4). There is no statistically significant difference in LNL levels between control and vehicle-treated rabbit corneas. The foregoing provides evidence to show the mechanism of action of copper eye drops on proteins in the collagen crosslinking pathway.

### Example 5—Combined Atropine and Copper Treatment Slows Progression of Myopia in Guinea Pigs

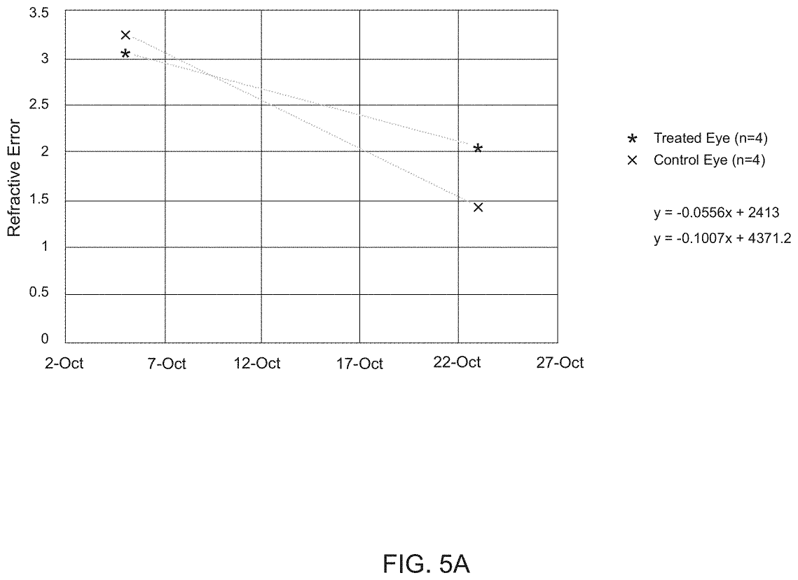

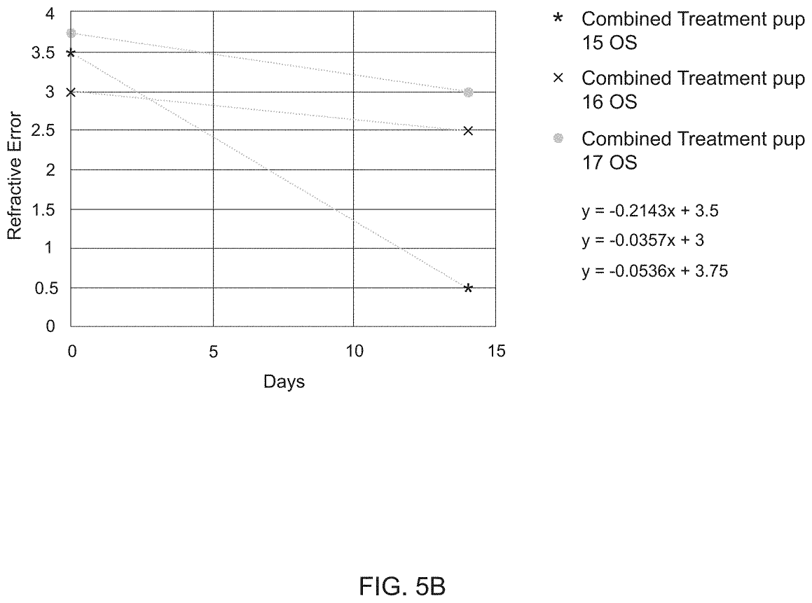

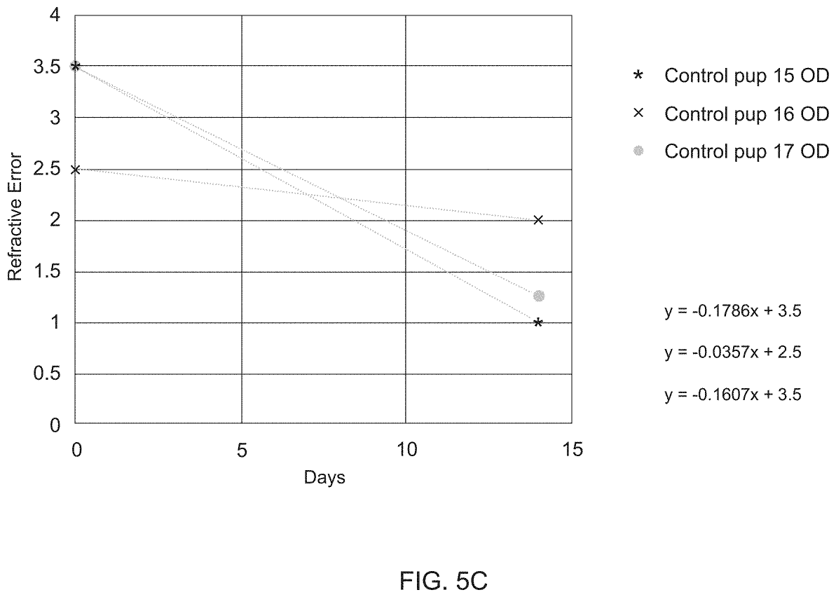

Treatment using Cu eye drops, or 0.001% atropine sulfate monohydrate eye drops with Cu, or control eye drops are administered at approximately 1 week of age to guinea pigs. The eye drops are administered 4 times per day for approximately 14 days. Refractive error is measured by an ophthalmologist prior to treatment on day 1 and at subsequent time-points (e.g., 14 days after the beginning of treatment) depicted in FIGS. 5A-5C. More specifically, refractive error is measured by streak retinoscopy in hand-held, awake animals in which cyloplegia has been previously induced with approximately 2 drops of 1% cyclopentolate. Stable refractive errors are typically obtained after 15 minutes when there is no pupil response. FIGS. 5A-5C present average refractive error measurements for the test subjects in each eye with linear fit.

It should be understood that the above-described methods are only illustrative of some embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that variations including, may be made without departing from the principles and concepts set forth herein.

What is claimed is:

1. A method of treating or preventing progression of myopia, comprising:
administering a therapeutically effective amount of an ophthalmic composition to an eye of a subject during a treatment period, said ophthalmic composition comprising:
a corneal cross-linking agent and a secondary therapeutic agent present in amounts sufficient to treat myopic progression; and
a pharmaceutically acceptable carrier.

2. The method of claim 1, wherein the ophthalmic composition is formulated as one of a solution, a suspension, an emulsion, a gel, a hydrogel, a thermo-responsive gel, a depot, a film, a gellating suspension, a contact lens, or a punctal plug.

3. The method of claim 1, wherein the ophthalmic composition is formulated as a sustained release composition that is configured to release copper-containing agent and the secondary therapeutic agent over a period of from about 2 days to about 6 months.

4. The method of claim 3, wherein administration is performed via one or more of placement of the composition in a cul-de-sac of the eye, placement of the composition in a conjunctival fornix of the eye, and placement of the composition in a sub-tenon's space of the eye.

5. The method of claim 3, wherein the ophthalmic composition is configured to deliver from about 0.0001 μg to about 5500 μg of copper per day to the eye of the subject, on average.

6. The method of claim 3, wherein the ophthalmic composition is configured to deliver from about 0.01 μg to about 200 μg of secondary therapeutic agent per day to the eye of the subject, on average.

7. The method of claim 1, wherein the secondary therapeutic agent is present in the composition in an amount from about 0.0005 wt % to about 2 wt %.

8. The method of claim 1, wherein the secondary therapeutic agent is present in the composition in an amount from about 0.001 wt % to about 1 wt %.

9. The composition of claim 1, wherein the amount of secondary therapeutic agent present in the composition is an amount from about 0.001 mg/ml to about 20 mg/ml.

10. The method of claim 1, wherein the secondary therapeutic agent is a member selected from the group consisting of atropine, homatropine, cyclopentolate, pirenzepine, 7-methylxanthanine, and combinations thereof.

11. The method of claim 1, wherein the corneal cross-linking agent is a copper-containing compound providing copper in an amount from about 0.000001 wt % to about 15 wt % of the composition.

12. The method of claim 1, wherein the corneal cross-linking agent is a copper-containing compound providing copper in an amount of from about 0.00001 mg/ml to about 1 mg/ml.

13. The method of claim 1, wherein the corneal cross-linking agent is a copper-containing compound providing copper in an amount of from about 0.015 mg/ml to about 0.15 mg/ml.

14. The method of claim 1, wherein the corneal cross-linking agent is a copper-containing compound selected from the group consisting of copper sulfate, copper carbonate, copper acetate, copper chloride, copper hydroxide, copper gluconate, copper bromide, copper fluoride, copper nitrate, copper iodide, copper perchlorate, copper molybdate, copper thiocyanate, copper tartrate, copper tetrafluoroborates, copper selenide, copper pyrophosphate, GHK-copper, tetra-amine copper sulfate, copper-histidine, copper-glycinate, and combinations thereof.

15. The method of claim 1, wherein the pharmaceutically acceptable carrier includes at least one of a tonicity agent, a solubilizing agent, a thickener, a polymer, a buffer, a preservative, a pH adjuster, and water.

16. The method of claim 1, wherein the composition has a tonicity of from about 200 mOsm/kg to about 600 mOsm/kg.

17. The method of claim 1, wherein the composition has a pH of from about 5.5 to about 8.5.

18. The method of claim 1, wherein the ophthalmic composition is formulated as an eye drop and carried in a container adapted to dispense the composition in a drop-wise manner at a drop volume of from about 5 μl to about 100 μl.

19. The method of claim 18, wherein the dosage form provides from about 0.0001 μg to about 500 μg of copper per drop of the ophthalmic composition.

20. The method of claim 1, wherein the composition is administered at from 1 to 4 time points per day per eye in need thereof.

21. The method of claim 20, wherein from about 5 μl to about 100 μl of the composition is administered at each time point.

22. The method of claim 1, wherein the composition is administered in connection with an ocular-shaping device configured to re-shape an elongated myopic eye.

23. The method of claim 1, wherein the subject is a human subject having an age of about 3 years to about 25 years.

24. The method of claim 1, wherein the treatment period is from about 6 months to about 5 years.

25. An ophthalmic composition for treating myopic progression, comprising:
an amount of a corneal cross-linking agent and an amount of a secondary therapeutic agent that are sufficient to treat myopic progression; and
a pharmaceutically acceptable carrier.

26. The composition of claim 25, wherein the ophthalmic composition is formulated as one of a solution, a suspension, an emulsion, a gel, a hydrogel, a thermo-responsive gel, a depot, a film, a gellating suspension, a contact lens, or a punctal plug.

27. The composition of claim 25, wherein the ophthalmic composition is formulated as a sustained release composition that is configured to release the corneal cross-linking agent and the secondary therapeutic agent over a period of from about 2 days to about 6 months.

28. The composition of claim 27, wherein the ophthalmic composition is configured to to deliver from about 0.0001 μg to about 500 μg of copper per day to the eye of the subject, on average.

29. The composition of claim 27, wherein the ophthalmic composition is configured to deliver from about 0.01 μg to about 200 μg of secondary therapeutic agent per day to the eye of the subject, on average.

30. The composition of claim 25, wherein the secondary therapeutic is present in an amount from about 0.0005 wt % to about 2 wt %.

31. The composition of claim 25, wherein the secondary therapeutic agent is present in an amount from about 0.001 wt % to about 1 wt %.

32. The composition of claim 25, wherein the amount of secondary therapeutic agent present in the composition is an amount from about 0.001 mg/ml to about 20 mg/ml.

33. The composition of claim 25, wherein the secondary therapeutic agent is a member selected from the group consisting of atropine, homatropine, cyclopentolate, pirenzepine, 7-methylxanthanine, and combinations thereof.

34. The composition of claim 25, wherein the corneal cross-linking agent is a copper-containing agent and is present in an amount of from about 0.000001 wt % to about 15 wt %.

35. The composition of claim 25, wherein corneal cross-linking agent is a copper-containing agent that provides an amount of copper to the composition of from about 0.00001 mg/ml to about 1 mg/ml.

36. The composition of claim 25, wherein corneal cross-linking agent is a copper-containing agent selected from the group consisting of copper sulfate, copper carbonate, copper acetate, copper chloride, copper hydroxide, copper gluconate, copper bromide, copper fluoride, copper nitrate, copper iodide, copper perchlorate, copper molybdate, copper thiocyanate, copper tartrate, copper tetrafluoroborates, copper selenide, copper pyrophosphate, GHK-copper, tetra-amine copper sulfate, copper-histidine, copper-glycinate, and combinations thereof.

37. The composition of claim 25, wherein the pharmaceutically acceptable carrier includes at least one of a tonicity agent, a solubilizing agent, a thickener, a polymer, a buffer, a preservative, a pH adjuster, and water.

38. The composition of claim 25, wherein the composition has a tonicity of from about 200 mOsm/kg to about 600 mOsm/kg. The composition of claim 25, wherein the composition has a pH of from about 5.5 to about 7.8.

40. The composition of claim 25, wherein the composition further comprises an additional active ingredient.

41. A topical ophthalmic dosage form, comprising:
an ophthalmic composition according to any one of claims 25-40 being formulated as an eye drop and carried in a container adapted to dispense the composition in a drop-wise manner at a drop volume of from about 5 μl to about 100 μl.

42. The dosage form of claim 41, wherein the dosage form provides from about 0.0001 μg to about 500 μg of copper per drop of the ophthalmic composition.

43. The dosage form of claim 41, wherein the dosage form provides from about 0.01 μg to about 200 μg of secondary therapeutic agent per drop of the ophthalmic composition.

44. The dosage form of claim 41, wherein the container is adapted to dispense the composition at a drop volume of from about 5 μl to about 100 μl.

45. Use of a corneal cross-linking agent and a secondary therapeutic agent in the preparation of an opthalmic medicament which when administered to an eye of a subject during a treatment period in a therapeutically effective amount treats myopic progression by at least increasing corneal lysyl oxidase activity.

46. The use of claim 45, wherein the ophthalmic medicament is formulated as one of a solution, a suspension, an emulsion, a gel, a hydrogel, a thermo-responsive gel, a depot, a film, a gellating suspension, a contact lens, or a punctal plug.

47. The use of claim 45, wherein the ophthalmic medicament is formulated as a sustained release composition that is configured to release a copper-containing agent and the secondary therapeutic agent over a period of from about 2 days to about 6 months.

48. The use of claim 47, wherein administration is performed via one or more of placement of the composition in a cul-de-sac of the eye, placement of the composition in a conjunctival fornix of the eye, and placement of the composition in a sub-tenon's space of the eye.

49. The use of claim 47, wherein the ophthalmic composition is configured to deliver from about 0.0001 μg to about 5500 μg of copper per day to the eye of the subject, on average.

50. The use of claim 47, wherein the ophthalmic composition is configured to deliver from about 0.01 μg to about 200 μg of secondary therapeutic agent per day to the eye of the subject, on average.

51. The use of claim 47, wherein the secondary therapeutic agent is present in the composition in an amount from about 0.0005 wt % to about 2 wt %.

52. The use of claim 45, wherein the secondary therapeutic agent is present in the composition in an amount from about 0.001 wt % to about 1 wt %.

53. The use of claim 45, wherein the amount of secondary therapeutic agent present in the composition is an amount from about 0.001 mg/ml to about 20 mg/ml.

54. The use of claim 45, wherein the secondary therapeutic agent is a member selected from the group consisting of atropine, homatropine, cyclopentolate, pirenzepine, 7-methylxanthanine, and combinations thereof.

55. The use of claim 45, wherein the corneal cross-linking agent is a copper-containing agent present in the composition in an amount from about 0.000001 wt % to about 15 wt %.

56. The use of claim 45, wherein the corneal cross-linking agent is a copper-containing agent that provides an amount of copper of from about 0.00001 mg/ml to about 1 mg/ml.

57. The use of claim 45, wherein the corneal cross-linking agent is a copper-containing agent selected from the group consisting of copper sulfate, copper carbonate, copper acetate, copper chloride, copper hydroxide, copper gluconate, copper bromide, copper fluoride, copper nitrate, copper iodide, copper perchlorate, copper molybdate, copper thiocyanate, copper tartrate, copper tetrafluoroborates, copper selenide, copper pyrophosphate, GHK-copper, tetra-amine copper sulfate, copper-histidine, copper-glycinate, and combinations thereof.

58. The use of claim 45, wherein the pharmaceutically acceptable carrier includes at least one of a tonicity agent, a solubilizing agent, a thickener, a polymer, a buffer, a preservative, a pH adjuster, and water.

59. The use of claim 45, wherein the medicament has a tonicity of from about 200 mOsm/kg to about 600 mOsm/kg.

60. The use of claim 45, wherein the medicament has a pH of from about 5.5 to about 8.5.

61. The use of claim 45, wherein the medicament further comprises an additional active ingredient.

62. The use of claim 45, wherein the ophthalmic medicament is formulated as an eye drop and carried in a container adapted to dispense the composition in a drop-wise manner at a drop volume of from about 5 μl to about 100 μl.

63. The use of claim 62, wherein the dosage form provides from about 0.0001 μg to about 500 μg of copper per drop of the ophthalmic composition.

64. The use of claim 45, wherein the medicament is administered at from 1 to 4 time points per day per eye in need thereof.

65. The use of claim 64, wherein from about 5 μl to about 100 μl of the composition is administered at each time point.

66. The use of claim 45, wherein the medicament is administered in connection with an ocular-shaping device configured to re-shape an elongated myopic eye.

67. The use of claim 45, wherein the subject is a human subject having an age of about 3 years to about 25 years.

68. The use of claim 45, wherein the treatment period is from about 6 months to about 5 years.

69. An ophthalmic composition for use in a method of treating or preventing progression of myopia, said method comprising:
administering a therapeutically effective amount of the ophthalmic composition to an eye of a subject during a treatment period, said ophthalmic composition comprising:
a corneal cross-linking agent and a secondary therapeutic agent present in amounts sufficient to treat myopic progression; and
a pharmaceutically acceptable carrier.

70. The ophthalmic composition for the use of claim 69, wherein the ophthalmic composition is formulated as one of a solution, a suspension, an emulsion, a gel, a hydrogel, a thermo-responsive gel, a depot, a film, a gellating suspension, a contact lens, or a punctal plug.

71. The ophthalmic composition for the use of claim 69, wherein the ophthalmic composition is formulated as a sustained release composition that is configured to release copper-containing agent and the secondary therapeutic agent over a period of from about 2 days to about 6 months.

72. The ophthalmic composition for the use of claim 71, wherein administration is performed via one or more of placement of the composition in a cul-de-sac of the eye, placement of the composition in a conjunctival fornix of the eye, and placement of the composition in a sub-tenon' s space of the eye.

73. The ophthalmic composition for the use of claim 71, wherein the ophthalmic composition is configured to deliver from about 0.0001 μg to about 5500 μg of copper per day to the eye of the subject, on average.

74. The ophthalmic composition for the use of claim 71, wherein the ophthalmic composition is configured to deliver from about 0.01 μg to about 200 μg of secondary therapeutic agent per day to the eye of the subject, on average.

75. The ophthalmic composition for the use of claim 69, wherein the secondary therapeutic agent is present in the composition in an amount from about 0.0005 wt % to about 2 wt %.

76. The ophthalmic composition for the use of claim 69, wherein the secondary therapeutic agent is present in the composition in an amount from about 0.001 wt % to about 1 wt %.

77. The ophthalmic composition for the use of claim 69, wherein the amount of secondary therapeutic agent present in the composition is an amount from about 0.001 mg/ml to about 20 mg/ml.

78. The ophthalmic composition for the use of claim 69, wherein the secondary therapeutic agent is a member selected from the group consisting of atropine, homatropine, cyclopentolate, pirenzepine, 7-methylxanthanine, and combinations thereof.

79. The ophthalmic composition for the use of claim 69, wherein the corneal cross-linking agent is a copper-containing compound providing copper in an amount from about 0.000001 wt % to about 15 wt % of the composition.

80. The ophthalmic composition for the use of claim 69, wherein the corneal cross-linking agent is a copper-containing compound providing copper in an amount of from about 0.00001 mg/ml to about 1 mg/ml.

81. The ophthalmic composition for the use of claim 69, wherein the corneal cross-linking agent is a copper-containing compound providing copper in an amount of from about 0.015 mg/ml to about 0.15 mg/ml.

82. The ophthalmic composition for the use of claim 69, wherein the corneal cross-linking agent is a copper-containing compound selected from the group consisting of copper sulfate, copper carbonate, copper acetate, copper chloride, copper hydroxide, copper gluconate, copper bromide, copper fluoride, copper nitrate, copper iodide, copper perchlorate, copper molybdate, copper thiocyanate, copper tartrate, copper tetrafluoroborates, copper selenide, copper pyrophosphate, GHK-copper, tetra-amine copper sulfate, copper-histidine, copper-glycinate, and combinations thereof.

83. The ophthalmic composition for the use of claim 69, wherein the pharmaceutically acceptable carrier includes at least one of a tonicity agent, a solubilizing agent, a thickener, a polymer, a buffer, a preservative, a pH adjuster, and water.

84. The ophthalmic composition for the use of claim 69, wherein the composition has a tonicity of from about 200 mOsm/kg to about 600 mOsm/kg.

85. The ophthalmic composition for the use of claim 69, wherein the composition has a pH of from about 5.5 to about 8.5.

86. The ophthalmic composition for the use of claim 69, wherein the ophthalmic composition is formulated as an eye drop and carried in a container adapted to dispense the composition in a drop-wise manner at a drop volume of from about 5 μl to about 100 μl.

87. The ophthalmic composition for the use of claim 86, wherein the dosage form provides from about 0.0001 μg to about 500 μg of copper per drop of the ophthalmic composition.

88. The ophthalmic composition for the use of claim 69, wherein the composition is to administered at from 1 to 4 time points per day per eye in need thereof.

89. The ophthalmic composition for the use of claim 88, wherein from about 5 μl to about 100 μl of the composition is administered at each time point.

90. The ophthalmic composition for the use of claim 69, wherein the composition is administered in connection with an ocular-shaping device configured to re-shape an elongated myopic eye.

91. The ophthalmic composition for the use of claim 69, wherein the subject is a human subject having an age of about 3 years to about 25 years.

92. The ophthalmic composition for the use of claim 69, wherein the treatment period is from about 6 months to about 5 years.
